(19) United States
(12) Patent Application Publication
(10) Pub. No.: US 2003/0208202 A1
(43) Pub. Date: Nov. 6, 2003

(54) Percutaneous screw fixation system

(76) Inventor: Mark H. Falahee, Ann Arbor, MI (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/427,445

(22) Filed: May 1, 2003

Related U.S. Application Data
(60) Provisional application No. 60/377,688, filed on May 4, 2002.

(51) Int. Cl. A61B 17/56

(52) U.S. Cl. 606/60

(57) ABSTRACT

A minimally invasive surgical screw fixation system and method are used in conjunction with fluoroscopy (i.e., C-arm), for percutaneous fixation of the facet joints of the spine through small incision ports. The approach is generally based on a right-triangle guidance system, positioned via the use of the C-arm and secured to the operative site. This particular configuration allows for percutaneous placement of screws across facet joints via known angles and pathways. The inventive instrumentation allows for the targeting of drill hole and, ultimately, the placement of the screws associated with the procedure.

### REFERENCE TO RELATED APPLICATION

This application claims priority from U.S. Provisional Patent Application Serial No. 60/377,688, filed May 4, 2002, the entire content of which is incorporated herein by reference.

### FIELD OF THE INVENTION

This invention relates generally to minimally invasive surgical procedures and, in particular, to a minimally invasive spinal fixation method and apparatus.

### BACKGROUND OF THE INVENTION

Minimally invasive surgical (MIS) procedures such as arthroscopic techniques have become increasingly popular. Compared to open surgical approaches, MIS procedures offer smaller incisions, less muscle stripping, and reduced operating-room recovery and hospitalization.

It has also been recognized that fixtures may be used in conjunction with certain types of spine-related surgical procedures to facilitate smaller incisions. As one example, U.S. Pat. No. 5,242,444 is directed to a method and device for percutaneous fixation and fusion of the lumbosacral vertebrae. Specifically, the approach involves the placement of instrumentation through the S1 pedicle to the vertebral body of L5 on both sides of the spine. A method of fusing or fixing the lumbosacral joint is carried out by placing instrumentation such as screws through the discovered bony pathways.

The instrumentation includes a device to guide placement of the instrumentation percutaneously using known radiographic techniques. The guide device may be connected to a localizing instrument, such as a pin anchored in the S1 pedicle. Once the device is connected to the localizing instrument, the shape of the device permits localization of the bony pathway leading to the vertebral body of L5.

Certain spinal fixation approaches involve fusion of the facet joints. A system for securing first and second opposing facet joints is disclosed in U.S. Pat. No. 5,527,312. The system is based on a cannulated drill having a side opening. An extendible member, received within the cannula includes a distal end which passes out of the side opening in a direction radially outwards from the axis of the cannulated drill as the extendible member is advanced distally through the cannula of the drill. The drill can be rotated such that the extendible member ablates opposite contacting surfaces of first and second facet joints, promoting fusion between the first and second facet joints. A facet screw passing through a hole drilled by the cannulated drill through the first facet joint and into the second facet joint is tightened into position to immobilize the first and second facet joints together.

A less invasive approach, described in U.S. Pat. No. 6,540,747, provides a system for stabilizing adjacent vertebra in the thoracolumbar and lumbar regions of the spine, and particularly, for fixing an inferior vertebra to a superior vertebra through the facet joint. The system includes a first facet screw extending through the inferior facet of the superior vertebra lateral to the sagittal plane and extending through the facet joint of the inferior vertebra into the base of the transverse process. A fixation bar is operably secured to the screw so as to prevent toggling of the screw relative to the bar. The bar has a finger that extends in a superior direction across the dorsal side of the transverse process of the superior vertebra. The finger continues by wrapping around the superior aspect of the pedicle of the superior vertebra and extends in an anterior direction, and preferably thereafter in an inferior direction, thus terminating in a hook-like structure that in combination with the screw fixes the structure in position. Preferably, a second screw and fixation bar are applied to the vertebra on the opposite side of the sagittal plane to provide bilateral fixation.

Despite such advances, there will always be a need for new protocols which are relatively simple in concept and execution, to ensure accurate and reproducible results despite differences in patient physiology and surgeon skill level.

### SUMMARY OF THE INVENTION

This invention resides in minimally invasive surgical screw fixation utilized to stabilize the spine. Used in conjunction with fluoroscopy (i.e., C-arm), the system and method allow for percutaneous fixation of the facet joints of the spine through small incision ports. The approach is generally based on a right-triangle guidance system, positioned via the use of the C-arm and secured to the operative site. This particular configuration allows for percutaneous placement of screws across facet joints via known angles and pathways. The inventive instrumentation allows for the targeting of drill hole and, ultimately, the placement of the screws associated with the procedure.

### BRIEF DESCRIPTION OF THE DRAWINGS

FIG. 1 is a simplified cross-section showing the way in which a target arm is passed to the lateral aspect of a facet joint associated with fixation;

FIG. 2 is a simplified cross-sectional drawing illustrating a right angle guide arm according to the invention and the way in which it is used;

FIG. 3 illustrates markers are lined up and fine-tuned, as necessary; and

FIG. 4 illustrates the fixation at one level.

### DETAILED DESCRIPTION OF THE INVENTION

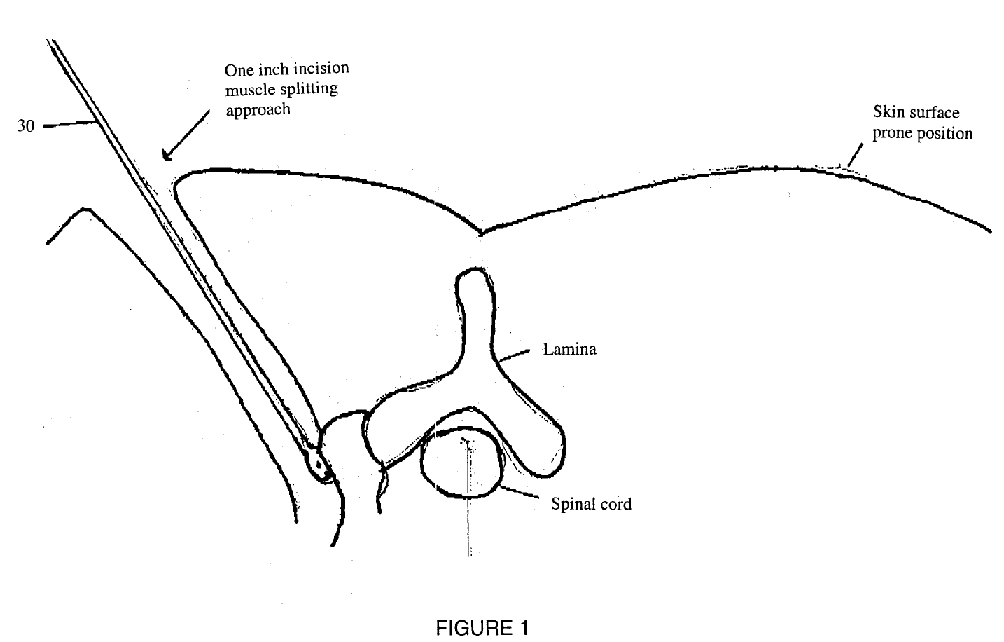

Reference is now made to the drawings, wherein FIG. 1 shows the way in which a 60-degree target arm 30 according to the invention is used to identify the lateral aspect of a facet joint to be fused. The patient is placed under generally anesthesia, in a prone position, and secured to a C-arm capable of operative (i.e., a Jackson table) to prevent patient movement or shift.

The C-arm is utilized in a posterior-anterior plane to assess location of an approximate one-inch incision for the 60-degree target arm 30. The site is prepped, draped, and the incision is made. A muscle-splitting finger dissection approach (i.e., via Wiltse) is used with respect to the lateral aspect of the facet joint. The 60-degree target arm 30 is passed along with the fingers to the lateral aspect of the joint and checked with the C-arm, as shown in FIG. 1.

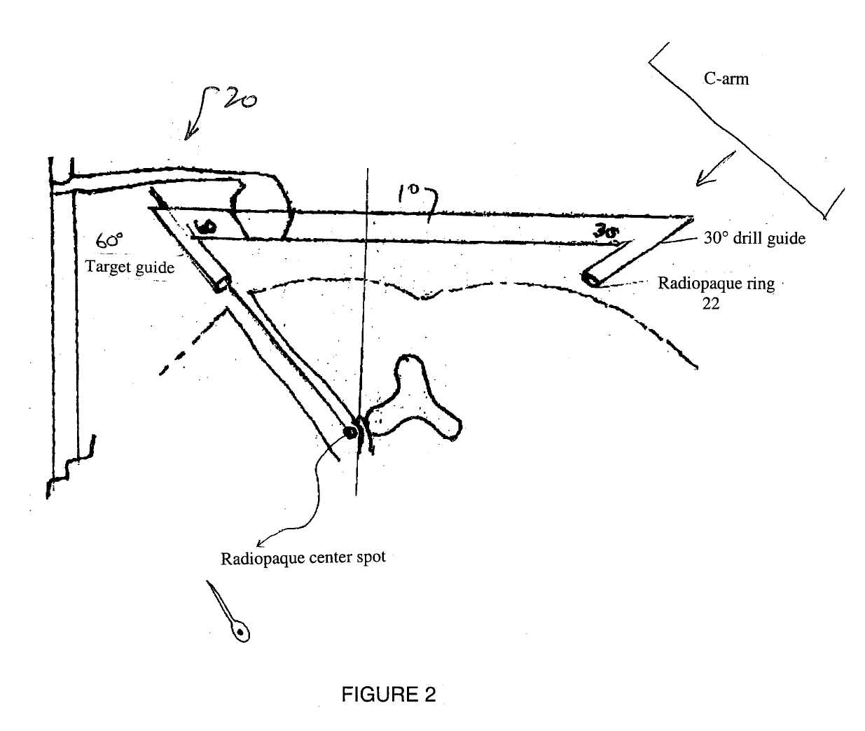

Now making to FIG. 2, a right-angle guidance system 10 according to the invention is attached at 20 to the operating table holding clamp of the Jackson table, to hold it into a stable stationary position. The guide is brought into position over the 60-degree target arm and temporarily secured. The position of the target arm tip is checked using the C-arm and palpation, as necessary.

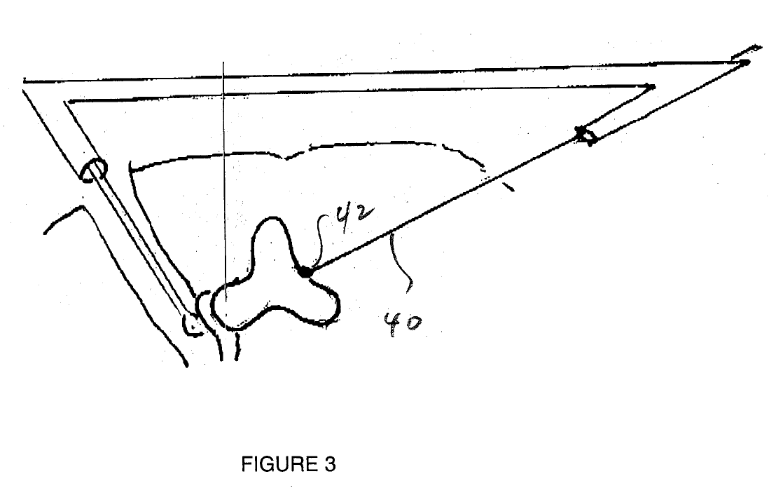

The assembly is held in position, with the guidance system secured to the operating room holding clamp and the arm secured to the guidance system, to achieve a rigid structure overall. The C-arm is aimed down the 30-degree drill guide and a radio-opaque ring 22 in the 30-degree arm is centered over the 60-degree target arm tip (see FIG. 2). The alignments are fine-tuned, as necessary. A blunt awl is journaled through the 30-degree target arm to mark the skin entry point, to provide for a puncture incision, and to tunnel through the muscle tissue to the lamina cortex. The blunt awl is replaced with a radioluscent target awl 40 having a marker 42 in its tip. The C-arm is used to ensure that the markers lined up, with fine-tuning being used as necessary, as shown in FIG. 3. The C-arm is then returned to the posterior-anterior (P-A) position.

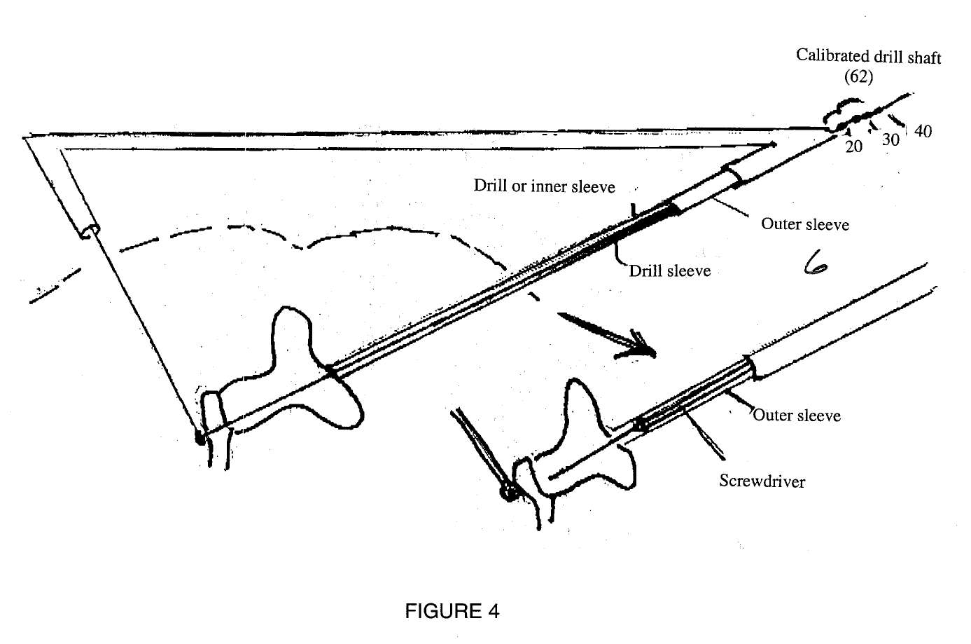

The 30-degree target awl 40 is replaced with outer sleeve and drill sleeve 60 under C-arm control, and passed through the 30-degree drill guide arm to the lamina cortex. A calibrated drill shaft 62 is used to check depth and select the appropriate screw length. A tap is made using the drill sleeve as necessary, after which the drill sleeve is removed and a screw is placed across the facet joint, as shown in FIG. 4.

The process is repeated by removing the sleeves, 60-degree target arm and rotating the guidance system out of position to mark the next level on the same side, rotated 180-degrees to approach from the opposite side. Note that, although a true right angle is used with respect to the inventive drill guide, including 60-degree and 30-degree portions, any triangulation system capable of producing the results desired and set forth herein may alternatively be used. All of the various components are preferably metal capable of sterilization and re-use, thought the invention is not limited in terms of applicable materials.

I claim:

1. A minimally invasive method of fixing a first bone to a second bone situated behind the first bone, comprising the steps of:
targeting a point behind the second bone;
providing a fixture including a guide defining a first axis;
orienting the guide so that the axis intersects the targeted point through the first bone; and
fastening the bones together with a fastener that penetrates through the first bone and at least a portion of the second bone along the axis.

2. The method of claim 1, wherein the bones form a facet joint.

3. The method of claim 1, wherein the step of targeting a point behind the second bone is carried out fluoroscopically using an elongated arm defining a second axis and terminating in a radio-opaque marker.

4. The method of claim 3, wherein the intersection of the first and second axes forms a right angle.

5. A minimally invasive method of fusing a facet joint, comprising the steps of:
targeting a point behind the facet joint;
providing a fixture including a guide defining a first axis;
orienting the guide so that the axis intersects the targeted point through a vertebra; and
fusing joint with a fastener aligned along the axis.

6. The method of claim 1, wherein the step of targeting a point behind the joint is carried out fluoroscopically using an elongated arm defining a second axis and terminating in a radio-opaque marker.

7. The method of claim 6, wherein the intersection of the first and second axes forms a right angle.

8. Instrumentation for fusing a first bone to a second bone situated behind the first bone forming a joint, comprising:
a fixture including an elongated member having first and second ends;
a first guide extending from the first end of the member along a first axis at a first angle relative to the member for targeting a point associated with the joint;
a second guide extending from the second end of the member along a second axis at a second angle relative to the member for fusing the joint; and
wherein the first and second axes intersect one another at a predetermined angle.

9. The instrumentation according to claim 8, wherein the predetermined angle is 90 degrees.

10. The instrumentation according to claim 8, wherein the first angle is 60 degrees, more or less.

11. The instrumentation according to claim 8, wherein the second angle is 30 degrees, more or less.

12. The instrumentation according to claim 8, wherein the bones form a facet joint.
